SEQUENCE LISTING (2 sequences)

SEQ ID NO: 1 - protein, 364 aa, Hepatitis C virus
MAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCNSMSTNPKPQKKNKRNTNRRPQDVKFPGGGQIVYLLPRRGPRLGVTRKTSERSQPRGRRQPIPKARRPEGRTWAQPGYPWPLYGNEGCGWAGWLLSP

SEQ ID NO: 2 - protein, 419 aa, Hepatitis C virus
MAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCNSMSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARA

(19) United States
(12) Patent Application Publication
(10) Pub. No.: US 2009/0068635 A1
(43) Pub. Date: Mar. 12, 2009

(54) INDIRECTLY LABELLED ASSAY CONJUGATES AND METHODS OF PREPARING AND USING SAME

(76) Inventors: Anthony S. Muerhoff, Kenosha, WI (US); Suresh M. Desai, Libertyville, IL (US); Thomas P. Leary, Kenosha, WI (US); George J. Dawson, Libertyville, IL (US); Robin A. Gutierrez, Gurnee, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/851,050

(22) Filed: Sep. 6, 2007

(51) Int. Cl. C12Q 1/70; C07K 1/107; C07K 14/765; G01N 33/53

(52) U.S. Cl. 435/5; 530/402; 530/409; 530/363; 435/7.1; 530/403

(57) ABSTRACT

Indirectly labelled assay conjugates prepared by a method that includes the step of submitting the binding member comprised by the conjugate to denaturing conditions prior to labelling the binding member. The indirectly labelled assay conjugates demonstrate an increased sensitivity when employed in diagnostic assays compared to assay conjugates prepared by methods that do not include a step of submitting the binding member to denaturing conditions prior to labelling. Processes for the preparation of the indirectly labelled assay conjugates, methods of detecting an analyte comprising the use of the indirectly labelled assay conjugate and kits comprising the indirectly labelled conjugates are also provided.

### FIELD OF THE INVENTION

The present invention pertains to the field of diagnostics and, in particular, to assay conjugates useful in diagnostic assays.

### BACKGROUND

Diagnostic assays, such as immunoassays, play an important role in a number of fields, including the medical and food safety fields. The sensitivity of diagnostic assays is an important feature of this type of assay and many methodologies have been developed in order to increase the sensitivity of such assays, for example, by improving signal generation or detection, or by reducing background.

As direct labelling of the detection reagents used in diagnostic assays can interfere with the ability of the detection reagent to bind to its target, indirect labelling techniques have been investigated as one approach for improving signal generation Indirect labelling generally involves the use of a linker or “spacer” molecule between the binding portion of the detection reagent (for example, an antibody, antigen or nucleic acid) and the detectable label.

Various improvements to further increase the sensitivity of reagents that are indirectly labelled have also been described. U.S. Pat. No. 4,994,385, for example, describes a polyamino acid based heterobifunctional coupling agent that includes a long, hydrophilic chain between the reactive ends of the agent which produces conjugates that may retain more of the activity of the conjugated protein than when shorter coupling agents are used. U.S. Pat. No. 5,853,723 also describes conjugates comprising long spacers. These conjugates comprise antibodies to glutamic acid decarboxylase coupled to detectable labels through a hydrophilic polymer, such as polyethylene glycol (PEG).

Indirect labelling can also allow for conjugation of the binding portion of a detection reagent to multiple labels through the use of a spacer molecule having multiple reactive groups and methods of increasing the numbers of labels conjugated to the detection reagent in order to increase the sensitivity of the reagent have been described. U.S. Pat. No. 5,656,426, for example, describes functionalized acridinium esters that can be conjugated to a detection reagent, such as an antibody or nucleic acid. Multiple labelling of the detection reagent with the esters is described, as is the use of a carrier molecule that is multiply labelled with the esters then conjugated to a nucleic acid probe.

The use of multiply labelled carrier molecules is also described in U.S. Pat. No. 4,975,532, which relates to the use of multiply labelled dextrans or Fab′ fragments of rabbit IgG as “bulking agents” or carriers that can be conjugated to a detection reagent Canadian Patent No. 1,330,061 describes a labelling reagent comprising avidin or streptavidin linked to a carrier that comprises 15 or more detectable labels. The labelling reagent can be linked via the avidin/streptavidin moiety to a biotin labelled assay component, such as an antibody, to provide a multiply labelled detection reagent suitable for use in a detection assay. U.S. patent application Ser. No. 10/172,944 (2003/0232386) and International Patent Application No PCT/US03/18954 (WO 03/106649) describe multiply labelled assay conjugates that comprise a heterophilic carrier labelled with at least 10 detectable labels, a detection reagent and a heterophilic linker that links the carrier to the detection reagent.

Urea is used in the purification of proteins (see, for example, U.S. Pat. No. 5,317,092). The use of urea to denature proteins and increase the number of groups available for labelling has also been described (see, for example, Smolka, M. B., et al. (2001) Anal Biochem. 297:25-31; Ramus, C., et al., (2006) Molecular & Cellular Proteomics 5:68-78; Exactag™ Labeling Kit (Perkin Elmer, Waltham, Mass.; International Patent Application No. PCT/US01/24279 (WO 02/16950) and U.S. patent application Ser. No. 11/249,683 (2006/0052279)). However, as treatment of proteins with urea is known to lead to unfolding of the protein and thus elimination of tertiary structure, as well as loss of helical structure and abolition of β-structure (see Bennoin, B. J. & Daggett, V, (2003) PNAS 100:5142-5147), use of urea in labelling reactions has been limited to contexts where there is no need to retain the three-dimensional structure of the protein in the labelled product, for example, when the end product is being prepared for analysis by mass spectrometry, electrophoresis or chromatography. Likewise, denaturation of proteins targeted by immunoassays has been described but is useful only when the target epitope is a linear epitope as opposed to a three-dimensional epitope (see U.S. Pat. No. 4,658,022). In addition, treatment of proteins with urea has been shown to increase the possibility of carbamylation of free cysteine residues (see Lippincott, J. & Apostol, I., (1999) Anal. Biochem. 267:57-64) leaving fewer reactive groups available for conjugation and/or reducing the biological activity of the protein (see U.S. patent application Ser. No. 10/785,369 (2004/166572)).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide indirectly labelled assay conjugates and methods of preparing and using the same. In accordance with one aspect of the invention, there is provided a process for preparing a protein conjugate, said process comprising the steps of: subjecting a protein to denaturing conditions to provide a treated protein; and conjugating said treated protein to a label moiety to provide said indirectly labelled protein conjugate, said label moiety comprising a carrier molecule coupled to one or more detectable labels.

In accordance with another aspect, there is provided a protein conjugate prepared by the process of the invention.

In accordance with another aspect, there is provided a method of detecting a target analyte in a sample comprising utilizing a protein conjugate of the invention, wherein said protein is capable of specifically binding said target analyte.

In accordance with another aspect, there is provided a kit comprising a protein conjugate of the invention and optionally instructions for use.

In accordance with another aspect of the inventions there is provided a protein conjugate comprising a hepatitis C virus NS3 antigen conjugated to a label moiety, said label moiety comprising a carrier moiety coupled to at least one detectable label.

### BRIEF DESCRIPTION OF THE FIGURES

These and other features of the invention will become more apparent in the following detailed description in which reference is made to the appended drawings.

FIG. 1 presents the amino acid sequence of the recombinant hepatitis C virus (HCV) antigen 9MB31 (SEQ ID NO:1), which includes amino acids 1192-1457 of HCV NS3 protein (underlined) fused to the first 150 amino acids of HCV core protein.

FIG. 2 presents a bar graph illustrating the sensitivity of conjugates comprising the recombinant hepatitis C virus antigen 9MB31 (SEQ ID NO:1) labelled with acridinium by various methods in detecting NS3 antibodies in a pool of human plasma known to contain antibodies to the core and NS3/4 regions (PC1) diluted as shown. Experiment No. refers to the experiments detailed in Tables 9 and 10.

FIG. 3 presents a bar graph illustrating the sensitivity of conjugates comprising the recombinant hepatitis C virus antigen 9MB31 (SEQ ID NO:1) labelled with acridinium by various methods in detecting NS3 antibodies in a pool of human plasma known to contain only antibodies against the NS3 region (PC2) using either a 1-step or 2-step assay format as indicated. Experiment No. refers to the experiments detailed in Tables 9 and 10.

FIG. 4 presents the amino acid sequence of the hepatitis C virus (HCV) antigen HC43 (SEQ ID NO:2) comprising amino acids 1192-1457 of NS3 fused at the C-terminus to amino acids 1-150 of the core protein (underlined).

### DETAILED DESCRIPTION OF THE INVENTION

The present invention provides for indirectly labelled assay conjugates prepared by a method that includes the step of submitting the binding member comprised by the conjugate to denaturing conditions prior to labelling the binding member. The indirectly labelled assay conjugates demonstrate an increased sensitivity when employed in diagnostic assays compared to assay conjugates prepared by methods that do not include a step of submitting the binding member to denaturing conditions prior to labelling. The indirectly labelled assay conjugates of the present invention preferably comprise a binding member conjugated to a carrier moiety that comprise one or more detectable labels. The assay conjugate can optionally further comprise a linker that conjugates the binding member and the carrier. Thus, in one embodiment, the invention provides for indirectly labelled assay conjugates having improved sensitivity. In another embodiment, the invention provides for a method of preparing the indirectly labelled assay conjugates. In another embodiment, the invention provides for a method of detecting an analyte comprising the use of the indirectly labelled assay conjugate. In some embodiments, the sensitivity of the method of detection may be adjusted by employing a 1-step or 2-step assay format. Kits comprising the indirectly labelled assay conjugates are also provided.

Definitions

Unless defined otherwise, all technical and scientific terms used herein have the same meaning as commonly understood by one of ordinary skill in the art to which this invention pertains,

The term “analyte,” as used herein, refers to a substance the presence, absence, or quantity of which is to be determined. Ate analyte can be a substance for which a naturally occurring binding member exists, or for which a binding member can be prepared. Non-limiting examples of analytes include, for example, antibodies, antigens, polynucleotides, polypeptides, proteins, hormones, cytokines, growth factors, steroids, vitamins, toxins, drugs (including those administered for therapeutic purposes as well as those administered for illicit purposes), and metabolites of the above substances, as well as bacteria, viruses, fungi, fungal spores and the like.

The term “antigen,” as used herein, refers to a molecule, a portion or portions of a molecule, or a combination of molecules or portions thereof up to and including whole cells, which is capable of inducing an immune response in an animal either alone or when conjugated to a suitable carrier molecule. An antigen may comprise a single epitope, or it may comprise a plurality of epitopes.

As used herein, the term “antibody” includes monoclonal antibodies and monospecific polyclonal antibodies, and both intact molecules as well as antibody fragments (such as, Fab, Fab′ and F(ab′)2, Fd, single-chain Fvs (scFv), single-chain antibodies, disulfide-linked Fvs (sdFv) and fragments comprising either a VL or VH domain) which are capable of specifically binding to a target analyte.

The term “test sample,” as used herein, refers to a sample that may contain the analyte of interest. For example, the test sample may be a biological fluid or tissue, such as whole blood or whole blood components (including red blood cells, white blood cells, platelets, serum and plasma), ascites, urine, cerebrospinal fluid, or other constituents of the body that may contain the analyte of interest, or the test sample may obtained from water, soil or vegetation, or the test sample may be a food sample or a swab taken from an area suspected of comprising the analyte of interest.

As used herein, the term “about” refers to approximately a ±10% variation from the stated value. It is to be understood that such a variation is always included in any given value provided herein, whether or not it is specifically referred to as such.

Components of the Indirectly Labelled Assay Conjugate

The assay conjugate of the present invention comprises a binding member conjugated to a carrier moiety comprising at least one detectable label. The assay conjugate can optionally further comprise a linker conjugating the carrier to the binding member.

Binding Member

In accordance with the invention, the binding member for inclusion in the conjugate is preferably a protein and is selected on the basis that it is capable of specifically binding to a target analyte of interest. In this context, the term “protein” includes full-length proteins (including proteins comprising leader sequences), mature proteins, protein fragments that retain the ability to specifically bind the target analyte (also referred to as “functional fragments”), and chimeric proteins.

Functional fragments may comprise a deletion of one or more amino acids from the N-terminus, the C-terminus, or the interior of the protein, or a combination thereof, provided that the fragments retain the ability to specifically bind the target analyte. In one embodiment of the invention in which the binding member is a functional fragment of an antigen, the functional fragment comprises at least one epitope recognizable by the antibody to be detected. In general, epitopes can be defined by a peptide of 5 amino acids or more in length. In one embodiment, therefore, the functional fragment is at least 5 amino acids in length. In another embodiment, the functional fragment is at least 8 amino acids in length. In other embodiments, the functional fragment is at least 9 amino acids in length, or at least 10 amino acids in length. In a further embodiment, the functional fragment is between about 5 amino acids and about 150 amino acids in length. (Contiguous amino acids are intended.)

In another embodiment, functional fragments are at least 25 amino acids in length. In a further embodiment, functional fragments aye at least 50 amino acids in length. In another embodiment, functional fragments are between about 25 and about 150 amino acids in length, for example between about 25 and about 140, between about 25 and about 130, between about 25 and 120, between about 25 and 110, or between about 25 and about 100 amino acids in length. In another embodiment, functional fragments are between about 50 and about 150 amino acids in length, for example between about 50 and about 140, between about 50 and about 130, between about 50 and 120, between about 50 and 110, or between about 50 and about 100 amino acids in length. (Again, contiguous amino acids are intended.)

A chimeric protein is formed by joining all or a part of the polypeptide sequences of two or more individual proteins. In one embodiment of the invention, the protein is a functional fragment or chimeric protein.

The binding member can be a naturally occurring protein, a recombinant protein, or a synthetic protein. Examples of suitable proteins include, but are not limited to, enzymes, antibodies, antigens, receptors, receptor ligands, hormones, growth factors, cytokines, and functional fragments and chimerics thereof Selection of an appropriate protein will be dependent on the intended use of the assay conjugate being prepared and can be readily determined by the skilled worker.

In one embodiment of the invention, the binding member is an antigenic protein. In the context of the present invention, an antigenic protein is a full-length protein (including a protein comprising a leader sequence), a mature protein, a functional fragment that retains the ability to specifically bind the target analyte, or a chimeric protein, which is capable of inducing an immune response in an animal, either alone or when conjugated to a suitable carrier molecule. An antigenic protein may comprise a single epitope, or it may comprise a plurality of epitopes. Examples of suitable antigenic proteins include, but are not limited to, bacterial proteins, viral proteins, prion proteins, tumour-associated antigenic proteins, and functional fragments and chimerics thereof.

In one embodiment, the binding member is a viral antigen. Examples of viral antigens include, but are not limited to, proteins from hepatitis A virus (HAV), hepatitis B virus (HBV), hepatitis C virus (HCV), herpes simplex virus (HSV), human immunodeficiency virus (HIV), rubella virus, cytomegalovirus (CMV), or West Nile virus (WNV). Suitable antigenic proteins from these viruses are known in the art and include, but are not limited to, HCV core protein, HCV E1 protein, HCV E2 protein, HCV NS3 protein, HCV NS4 protein, HCV NS5 protein, HBV HBsAg antigen, HBV core protein, HIV envelope protein and HIV p24. Functional fragments and chimeras of these proteins are also suitable. In one embodiment, the binding member is a HCV antigen or functional fragment, or an HCV chimeric protein.

In another embodiment, the binding member is a bacterial antigen. Examples of bacterial antigens include, but are not limited to, antigens derived from Helicobacter pylori. In another embodiment, the binding member is an antigen derived from a parasite. Examples of parasitic antigens include, for example, antigens derived from Trypanosoma cruzi, Plasmodium falciparum, Plasmodium vivax, Plasmodium ovale, Plasmodium malariae, or Toxoplasmosis gondii.

Carrier Moiety

The assay conjugate further comprises a carrier moiety. The carrier moiety is conjugated to at least one detectable label, therefore, suitable carrier moieties are those that comprise reactive groups for conjugation with at least one detectable label, or that can be derivatized to comprise such reactive groups. Examples of such reactive groups include sulphydryl groups, amines, carboxyls, and the like. In one embodiment of the invention, the carrier moiety comprises at least one reactive group allowing conjugation to at least one detectable label. Examples of suitable carrier moieties include, but are not limited to, synthetic materials such as, for example, latex, polyoxyethylene or polyethylene glycol; polysaccharides, such as, for example, derivatized dextran or cyclodextran; and proteins such as, for example, bovine serum albumin, thyroglobulin, haemocyanin, myosin, apoferritin, ovalbumin or α₂-macroglobulin.

The carrier moiety can be directly attached to the binding member, or it can be attached via an appropriate linker. Various linkers suitable for this purpose are known in the art and described in more detail below.

In one embodiment of the invention, the carrier moiety comprises at least one detectable label. In one embodiment, the carrier molecule comprises between 2 and about 15 detectable labels. In another embodiment, the carrier molecule comprises between 3 and about 15 detectable labels. In another embodiment, the carrier molecule comprises between 4 and about 15 detectable labels. In other embodiments, the carrier molecule comprises between 5 and about 15, 5 and about 13, 5 and about 12, 5 and about 11 or 5 and about 10 detectable labels. In a specific embodiment, the carrier molecule comprises about 8 detectable labels.

Detectable Label

The assay conjugate further comprises one or more detectable labels conjugated to the carrier moiety. Detectable labels are molecules or moieties a property or characteristic of which can be detected directly or indirectly. Selection of an appropriate detectable label can be readily made by the skilled technician based on, for example, the properties of the carrier moiety to be labelled and the intended end use of the assay conjugate.

Examples of detectable labels include, but are not limited to, radioisotopes (e.g., ³H, ¹⁴C, ³⁵S, ¹²⁵I, ¹³¹I), fluorescent labels (e.g., FITC, rhodamine, lanthanide phosphors, lanthanide chelates, fluorescein isothiocyanate, phycoerytherin, phycocyanin, allophycocyanin, o-phthaldehyde, fluorescamine, and commercially available fluorophores such as Alexa Fluor 350, Alexa Fluor 488, Alexa Fluor 532, Alexa Fluor 546, Alexa Fluor 568, Alexa Fluor 594, Alexa Fluor 647, and BODIPY dyes such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine and Texas Red), enzymatic labels (for example, horseradish peroxidase, β-galactosidase, β-lactamase, alkaline phosphatase), chemiluminescent groups (for example, acridinium compounds, such as acridinium esters, acridinium sulphonamides, and acridinium salts; luminol; isoluminol; phenanthridiniums; 1,2-dioxetanes; imidazoles, and oxalate esters), bioluminescent groups (for example, luciferin, luciferase and aequorin), paramagnetic labels (for example, chromium (III), manganese (II), manganese (III), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III)) which can be detected by MRI, predetermined polypeptide sequences recognised by a secondary reporter (for example, leucine zipper pair sequences, binding sites for secondary antibodies, metal binding domains, epitope tags), and the like. One skilled in the art will understand that detectable labels may require additional components, such as substrates, triggering reagents, light, and the like to enable detection of the label. In some embodiments, detectable labels are attached by spacer arms of various lengths to reduce potential steric hindrance.

In one embodiment of the invention, the detectable label incorporated into the assay conjugate is a chemiluminescent group. In another embodiment, the detectable label incorporated in to the assay conjugate is an acridinium compound.

In another embodiment, the detectable label incorporated into the assay conjugate is an enzymatic label. In a further embodiment, the detectable label incorporated into the assay conjugate is horseradish peroxidase.

Methods of labelling various carrier moieties are well-known in the art (see, for example, Ausubel et al., (1997 & updates) Current Protocols in Molecular Biology, Wiley & Sons, New York; Harlow et al., Antibodies: A Laboratory Manual, Cold Spring Harbor Laboratory Press, 2nd ed. 1988); Coligan et al., Current Protocols in Protein Science, Wiley & Sons, New York). The detectable label can be bound to the carrier moiety either directly or though a coupling agent such as, for example, EDAC (1-ethyl-3-(3-dimethylaminopropyl)carbodiimide, hydrochloride). Other coupling agents that can be used are known in the art. In addition, detectable labels that already contain end groups that facilitate the coupling of the detectable label to the carrier can be purchased or synthesised, for example, N10-(3-sulphopropyl)-N-(3-carboxypropyl)-acridinium-9-carboxamide (or CPSP-acridinium ester) or N10-(3-sulphopropyl)-N-(3-sulphopropyl)-acridinium-9-carboxamide (or SPSP-acridinium ester)

Linker

The assay conjugate may optionally comprise a linker that conjugates the labelled carrier to the binding member. Linkers are bifunctional moieties that serve to covalently connect the carrier to the binding member. The linker comprises at least two functionalities, one for attaching to the carrier and the other for attaching to the binding member. The functionalities can be the same (homobifunctional linker) or different (heterobifunctional linker). Non-limiting examples of linker functionalities include amino, hydroxyl, carboxylic acid, thiol, phosphoramidate, phosphate, phosphite, unsaturations (for example, double or triple bonds), and the like.

Various linkers are known in the art and many are commercially available (for example, from Pierce Chemical Co., Rockford, Ill.). Examples of linkers include, but are not limited to, succinimide-based linkers, such as succinimidyl 4-(N-maleimidomethyl)cyclohexane-1-carboxylate (SMCC), succinimidyl 4-(N-maleimidomethyl)cyclo-hexane-1-carboxy-(6-amido-caproate) (LCSMCC), succinimidyl m-maleimido-benzoylate (MBS), succinimidyl N-e-maleimido-caproylate (EMCS), succinimidyl 6-(β-maleimido-propionamido) hexanoate (SMPH), succinimidyl 4-(N-maleimido acetate) (AMAS) and succinimidyl 4-(p-maleimidophenyl)butyrate (SMPB); 6-aminohexanoic acid (AHEX or AHA); 8-amino-3,6-dioxaoctanoic acid (ADO); 6-aminohexyloxy; 4-aminobutyric acid; 4-aminocyclohexylcarboxylic acid; β-alanine; phenylglycine (PHG); 4-aminocyclohexanoic acid (ACHC); β-(cyclopropyl)alanine (β-CYPR); amino dodecanoic acid (ADC); alylene diols, polyethylene glycols, amino acids, and the like. Tri-functional crosslinking agents such as tris-(2-maleimidoethyl)amine and tris-(succinimidyl amino-triacetate) are also commercially available (for example, from Pierce Chemical Co., supra).

In one embodiment of the invention, the indirectly labelled conjugate comprises a carrier moiety conjugated to the binding member by a linker. In one embodiment, the indirectly labelled conjugate comprises a carrier moiety conjugated to the binding member by a succinimide based linker. In a specific embodiment, the indirectly labelled conjugate comprises a carrier moiety conjugated to the binding member by a LCSMCC linker,

Process of Preparing Indirectly Labelled Assay Conjugates

The process of preparing indirectly labelled assay conjugates according of the invention comprises the steps of subjecting the selected binding member to denaturing conditions and subsequently conjugating the binding member to a label moiety which comprises a carrier moiety coupled to one or more detectable labels.

Conditions that will fully or partially denature proteins, such as the binding members contemplated by the present invention, are well known in the art. Examples of denaturing conditions include, but are not limited to, an increase in temperature; a substantial increase or decrease in the pH relative to the pH of the native environment in which the protein is functional; addition of a protein denaturing agent; addition of an organic solvent; addition of a high concentration of a salt relative to the salt concentration of the native environment in which the protein is functional, or a combination of two or more of these conditions.

The exact denaturing conditions used will be dependent in part on the protein being employed and can be readily determined by those of skill in the art. For example, when an increase in temperature is being employed as a denaturing condition, an increase in temperature to between about 37° C. and about 60° C. is appropriate for most proteins derived from eukaryotes or mesophilic prokaryotes. For example, the protein can be submitted to denaturing conditions comprising a temperature between about 45° C. and about 60° C. Denaturing pH conditions can be, for example, a pH of more than about pH 8 or less than about pH 6. pH increases may be effected by addition of a suitable organic or inorganic base to the protein, for example. Tris base, ammonia, sodium hydroxide, sodium carbonate and the likes pH decreases can be effected by addition of an appropriate organic or inorganic acid to the protein, for example, acetic acid, hydrochloric acid, sulphuric acid, phosphoric acid and the like.

When a protein denaturing agent is employed, the agent can be selected from various protein denaturing agents known in the art. Examples of protein denaturing agents include, but are not limited to, urea; thiourea; guanidinium hydrochloride; chaotropic agents such as a thiocyanate salt (for example, KSCN or NH₄SCN); and detergents such as sodium dodecyl sulphate, sodium deoxycholate, a polyoxyethylene alcohol (for example, the Brij series of detergents), an octoxynol (for example, the Triton series of detergents) or Tween 20. Detergents can be used at concentrations of about 0.01% to about 10% by volume to create denaturing conditions. Other denaturing agents can be used at concentrations between about 0.5M and about 10M. For example, urea or thiourea can be used at concentrations between about 2M and about 10M, for example between about 3M and about 10M, between about 4M and about 10M, or between about 5M and about 10M. In one embodiment, the process of preparing the indirectly labelled assay conjugate comprises subjecting the binding member to denaturing conditions that comprise treatment with about 5M to about 9M urea or thiourea.

Examples of suitable organic solvents for creating denaturing conditions include, but are not limited to, methanol, ethanol, propanol, 2-propanol, dimethyl formamide, acetonitrile, polyethylene glycol, glycol, glycerol, phenol and 1,4-butane diol. The organic solvent can be added in an amount of for example, up to about 95% by volume depending on the type of solvent.

When a salt is used to create denaturing conditions, it may suitably be used at a concentration of about 0.5M to about 6M, for example between about 1M and about 3M. Examples of salts which are useful in this context include, but are not limited to, NaCl, LiCl, MgCl₂, (NH₄)₂SO₄, NaOAc, K₂SO₄, KOAc, sodium phosphates and sodium citrates,

The binding member can be subjected to one or more of the above-noted denaturing conditions, for example, by increasing the temperature of a solution comprising the binding member; by dialysing a solution of the binding member against a second solution having an increased or decreased pH, or comprising an appropriate concentration of a denaturing agent or salt; by exposing an immobilized form of the binding member (for example, on a chromatography column or other solid support) to a solution having an increased or decreased pH, or comprising an appropriate concentration of a denaturing agent or salt, or a combination of the above. Other techniques known in the art can also be employed.

Subsequent to the denaturing step, various methods of conjugation known in the art can be employed to conjugate the binding member to the labelled carrier moiety (see, for example, “Bioconjugation: Protein Coupling Techniques for the Biomedical Sciences” Edited by M. Alsam and A. Dent, Macmillan Reference Ltd., London, 1998). The selection of the method will be dependent on the binding member and carrier being employed and can be readily determined by one of skill in the art. As noted above, the binding member can be conjugated directly with the labelled carrier or it can be conjugated via a linker. The linker can be pre-attached to the carrier or the binding member by art known methods prior to addition of the other reaction component, or it can be added to the reaction mixture together with the carrier and the binding member. Where appropriate, the binding member can be treated with a suitable reagent to expose reactive groups in the binding member for conjugation as is known in the art, for example, the binding member can be treated with a reducing agent, such as dithiothreitol (DTT), dithioerythritol, mercaptoethanol, cysteine, decarboxy-cysteine or tris(2-carboxyethyl)phosphine (TCEP) to reduce sulphydryl groups. The binding member may also be treated with a derivatising agent to create reactive groups for reaction with the carrier moiety by art-known methods.

In one embodiment, the process of the present invention comprises exposing the binding member to denaturing conditions followed by treatment with a reducing agent prior to reaction with the labelled carrier moiety.

When the denaturing conditions comprise treatment of the binding member with a denaturing agent or salt or exposure to high or low pH, the binding member can be subjected to an additional step prior to conjugation to reduce the amount of denaturing agent/salt present in the preparation or to adjust the pH of the preparation. For example, the binding member can be subjected to one or more purification steps to remove all or a portion of the denaturing agent/salt or to adjust the pH, or it may be subjected to a dilution step to decrease the concentration of the denaturing agent/salt or to adjust the pH. For example, the binding member can be dialysed against a suitable buffer, subjected to chromatography, passed through a desalting column prior to conjugation, or simply diluted by addition of a suitable buffer.

In one embodiment, the method of the invention comprises subjecting the binding member to denaturing conditions that comprise treatment with a denaturing agent and subsequently reducing the concentration of the denaturing agent for conjugation with the carrier. In one embodiment of the invention, the dilution is carried out in the reaction buffer for the conjugation reaction such that the final amount of denaturing reagent in the conjugation reaction has been reduced to between about 92% and about 0.5% of the initial concentration. In one embodiment, the process comprises reducing the concentration of the denaturing agent to between about 60% and about 0.5% of the initial concentration. In another embodiment, the process comprises reducing the concentration of the denaturing agent to between about 30% and about 0.5% of the initial concentration. In a further embodiment, the process comprises reducing the concentration of the denaturing agent to between about 12% and about 0.5% of the initial concentration. In other embodiments, the process comprises reducing the concentration of the denaturing agent to between about 18% and about 0.5%, between about 16% and about 0.5%, between about 15% and about 0.5%, or between about 12% and about 0.5% of the initial concentration.

In a specific embodiment of the invention in which urea is used as the denaturing agent, the dilution is carried out in the reaction buffer for the conjugation reaction such that the final amount of urea in the conjugation reaction has been reduced to between about 20% and about 0.5% of the initial concentration, for example, between about 15% and about 0.5%.

The ratio of binding member and labelled carrier to be included in the conjugation reaction will depend on the conjugation technique being employed, as well as the identity of the binding member and the carrier and can be readily determined by a worker slcilled in the art. In one embodiment, the binding member and the labelled carrier can be reacted in a molar ratio between about 10:1 and about 1:10, for example, between about 8:1 and about 1:8, or between about 5:1 and about 1:5. In one embodiment, the process comnprises reacting the binding member and the multiply labelled carrier together in a molar ratio between about 1:1 and about 1:5.

Characterization of the Indirectly Labelled Assay Conjugate

The ability of the indirectly labelled assay conjugate to detect its target analyte can be tested by standard techniques. In accordance with one embodiment of the invention, the indirectly labelled assay conjugate has an increased sensitivity compared to an analogous conjugate prepared without submitting the binding member to a denaturing step. The relative sensitivities of the two conjugates can be assessed by art known methods using test samples known to contain the analyte of interest. (The Immunoassay Handbook, Second Edition, Edited by David Wild, Nature Publishing group, London, UK, 2001.)

In the context of the present invention, the indirectly labelled assay conjugate is considered to demonstrate an increased sensitivity if it exhibits an increase in signal-to-negative ratio of at least about 10% compared to the analogous conjugate that has not been submitted to a denaturing step. In one embodiment, the indirectly labelled assay conjugate exhibits an increase in signal-to-negative ratio of at least about 20% compared to the analogous conjugates. In another embodiment, the indirectly labelled assay conjugate exhibits an increase in signal-to-negative ratio of at least about 30% compared to the analogous conjugate. In a further embodiment, the indirectly labelled assay conjugate exhibits an increase in signal-to-negative ratio of at least about 40% compared to the analogous conjugate. In other embodiments, the indirectly labelled assay conjugate exhibits an increase in signal-to-negative ratio of at least about 50%, at least about 60%, at least about 70%, at least about 80%, at least about 90%, or at least about 100% compared to the analogous conjugate.

Uses

The process of the invention can be used to prepare indirectly labelled assay conjugates that have application in a number of settings where detection of an analyte is required. For example, the assay conjugates can be used in a clinical context for detection of viruses or bacteria, or antibodies raised against a virus, bacteria or other infection, or for diagnostic purposes based on the detection of antibodies, antigens, proteins, hormones and the like. The assay conjugates can also be used in a food safety context for the detection of microorganisms or contaminating chemicals in foodstuff, as well as in environmental settings for the detection of microorganisms or contaminants in soil, water or other materials, and in hospital or other settings where detection of the presence of a particular analyte, for example, on surfaces or instruments, may be important.

In one embodiment of the present invention, the process can be employed to prepare assay conjugates for use in the detection of an analyte that is an antibody or an antigen in a test sample, for example, in an immunoassay context. In this context, the assay conjugate can be used as a direct detection reagent for detection of an antibody or antigen, or as a secondary detection reagent that allows detection of an antigen trough an intermediary antibody detection agent.

For example, the assay conjugate can be used in a direct assay method for detecting a target analyte, such as an antibody, which method comprises the steps of: contacting a sample known to comprise, or suspected of comprising, the target analyte with the assay conjugate under conditions that allow the binding of the assay conjugate to the target analyte to form a complex, and detecting the complex as an indication of the presence of target analyte in the sample. In one embodiment of the invention, the assay conjugate is utilised in a direct assay method.

An example of an indirect assay method for detecting a target analyte, such as an antibody or antigen, would employ a binding agent that is capable of binding a target analyte and an assay conjugate that is capable of specifically binding the binding agent and would comprise the steps of: contacting a sample known to comprise, or suspected of comprising, the target analyte with the binding agent under conditions that allow binding of the binding agent to the target analyte to form a complex, contacting the complex with the assay conjugate under conditions that allow binding of the assay conjugate to the binding agent, and detecting bound assay conjugate as an indication of the presence of target analyte in the sample

The assay employing the indirectly labelled assay conjugate can also employ an immobilised capture agent that specifically binds to the target analyte. The capture agent can be the same as the binding member comprised by the assay conjugate or it can be different. The assay conjugate can thus be used in a sandwich assay format as is known in the art. The capture agent can be immobilised on a suitable solid support. Various suitable solid supports are known in the art (see, for example, Current Protocols in Protein Science, Coligan, J. E., et at. (eds.), John Wiley & Sons, (2005 & updates); Affinity Chromatography: Principles & Methods, Pharmacia LKB Biotechnology (1988), and Doonan, Protein Purification Protocols, The Humana Press (1996)). Examples include, but are not limited to, various resins and gels (such as silica-based, cellulosic, cross-linked polyacrylamide, dextran, agarose or polysaccharide resins or gels), membranes (such as nitrocellulose or nylon membranes), beads and microparticles (such as those made of glass, agarose, cross-linked agarose, polystyrene, various magnetic materials, polyacrylamide, latex and dimethylacrylamide), chitin, sand, pumice, glass, metal, silicon, rubber, polystyrene, polypropylene, polyvinylchloride, polyvinyl fluoride, polycarbonate, latex, diazotized paper, and the like that are insoluble under the conditions in which they are to be used.

The solid support can be particulate (pellets, beads, and the like), as noted above, or can be in the form of a continuous surface (membranes, meshes, plates including multi-well plates, slides, disks, capillaries, hollow fibres, needles, pins, chips, solid fibres, gels, and the like). The solid supports can be modified as necessary with reactive groups that allow attachment of the capture reagent by amino groups, carboxyl groups, sulphydryl groups, hydroxyl groups and/or carbohydrate moieties. Examples of coupling chemistries that can be employed to immobilise the capture reagent on the solid support include cyanogen bromide activation, N-hydroxysuccinimide activation, epoxide activation, sulphydryl activation, hydrazide activation, and carboxyl and amino derivatives for carbodiimide coupling chemistries. Alternatively, the capture agent may be modified with a group that allows for attachment to an appropriately modified solid support. For example, the capture agent can comprise a His-tag that allows for immobilisation on a solid support that has been modified to contain Ni²⁺ ions. Other examples are known in the art.

An example of a method for a sandwich assay comprises the steps of: (a) contacting an immobilized capture agent capable of specifically binding to a target analyte with a sample known to comprise, or suspected of comprising, the target analyte under conditions that allow the binding of the target analyte to the capture agent to form a first complex, (b) contacting said first complex with the assay conjugate under conditions that allow binding of the assay conjugate to the target analyte such that a second complex comprising the binding member, target analyte and assay conjugate is formed, and (c) detecting the second complex as an indication of the presence of target analyte in the sample. As is known in the art, steps (a) and (b) may be conducted concurrently or sequentially. In one embodiment of the invention, the indirectly labelled assay conjugate is employed in a sandwich assay for detection of a target analyte. In another embodiment, the indirectly labelled assay conjugate is employed in a sandwich assay which employs a capture agent that is different to the binding member comprised by the assay conjugate.

The method can also be applied in a competitive assay format or a non-competitive assay format. For example, the method can be a competitive assay that employs an immobilised capture agent capable of specifically binding both a target analyte and the assay conjugate, the method comprising the steps of: contacting the immobilized capture agent with the assay conjugate and a sample known to comprise, or suspected of comprising, the target analyte under conditions that allow the binding of the assay conjugate and the target analyte to the capture agent, and detecting the presence of complexes comprising the capture agent and assay conjugate as an indication of the presence of target analyte in the sample.

Alternatively, the method can be a non-competitive assay method An example of a non-competitive sandwich assay is provided above. In one embodiment of the invention, the assay conjugate is utilised in a noncompetitive assay

In one embodiment, the process can be employed to prepare assay conjugates for use in the detection of an analyte that is indicative of the presence of an infectious disease. For example, the assay conjugate can be used in the detection of antigens from or antibodies to cytomegalovirus (CMV), rubella virus, hepatitis A virus (HAV), hepatitis B virus (HBV), hepatitis C virus (HCV), herpes simplex virus (HSV) or human immunodeficiency virus (HIV), West Nile virus (WNV), infectious particles indicative of bovine spongiform encephalopathy (BSE), Helicobacter pylori, Trypanosoma cruzi, Plasmodium falciparum, Plasmodium vivax, Plasmodium ovale, Plasmodium malariae or Toxoplasmosis gondii. In a specific embodiment, the assay conjugates are for use in the detection of an antibody raised against, or an antigen from, a virus. Examples of suitable binding members in this context include, but are not limited to, HCV core protein, HCV NS3 protein, HBV HBsAg antigen and HIV p24, as well as functional fragments of and chimeras derived from these proteins,

In one embodiment, the process of the invention can be employed to prepare assay conjugates for use in the detection of an analyte that is indicative of the presence of a non-infectious disease, such as cancer. For example, the assay conjugate can be used in the detection of antibodies to thyroglobulin (Tg) or thyroid peroxidase (TPO).

The assay conjugates produced by the process of the invention are suitable for use in “high-throughput” assays. In general, high-throughput assays employ a capture agent that has been immobilised onto a solid support and a detection reagent (i.e. an assay conjugate) that is either a free reagent or immobilized on a second solid support. Various suitable solid supports are known in the art, as described above.

High-throughput assays provide the advantage of processing a plurality of samples simultaneously and thus significantly decrease the time required to screen a large number of samples. For high-throughput screening, assay components are usually housed in a multi-container carrier or platform, such as multi-well plates, which allows a plurality of assays to be monitored simultaneously. Many high-throughput assay systems are available commercially, as are automation capabilities for many procedures such as sample and reagent pipetting, liquid dispensing, timed incubations, formatting samples into a high-throughput format and microplate readings in an appropriate detector, resulting in much faster throughput times.

Of course, it goes without saying that any of the exemplary formats herein, and any assay or kit according to the invention can be adapted or optimized for use in automated and semi-automated systems (including those in which there is a solid phase comprising a microparticle), as described, e g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits of the present invention optionally can be adapted or optimized for point of care assay systems, including Abbott's Point of Care (i-STAT™) electrochemical immunoassay systems. Immunosensors and methods of manufacturing and operating them in single-use test devices are described, for example in U.S. Pat. No. 5,063,081 and published US Patent Applications 20030170881, 20040018577, 20050054078, and 20060160164 (incorporated by reference herein for their teachings regarding same).

Assay Kits

The invention further provides for assay kits comprising one or more indirectly labelled assay conjugates of the invention. The assay conjugates can be provided in the kits as solutions or lyophilised preparations, or they may be immobilised on a suitable solid support, such as those described above.

The kits can further optionally comprise reagents to facilitate conducting the assay, such as capture reagents, diluents, buffers, salts, enzymes, enzyme co-factors, substrates, detection reagents, and the like. Other components to facilitate the isolation of and/or for treatment of a test sample, such as buffers and diluents, may also be included in the kit. The kit may additionally include one or more controls. One or more of the components of the kit may be lyophilised and the kit may further comprise reagents suitable for the reconstitution of the lyophilised components.

The various components of the kit are provided in suitable containers. One or more of the containers may be a microtitre plate. Where appropriate, the kit may also optionally contain reaction vessels, mixing vessels and other components that facilitate the preparation of reagents or the test sample. The kit may also include one or more instruments for assisting with obtaining or handling a test sample, such as a syringe, pipette, forceps, measured spoon, or the like.

The kit can optionally include instructions for use, which may be provided in paper form or in computer-readable form, such as a disc, CD, DVD or the like,

In one embodiment, the present invention provides for a kit for the detection of HCV comprising an indirectly labelled assay conjugate of the invention that specifically binds to a HCV antibody. In another embodiment, the present invention provides for a kit for the detection of HCV comprising an indirectly labelled assay conjugate that specifically binds to a HCV antibody and is immobilised on microparticles.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Automated Magnetic Microparticle-Based Immunoassay

The conjugates prepared as described in the following Examples were tested for their ability to detect anti-HCV NS3 antibodies using an automated immunoanalyzer that utilizes paramagnetic microparticles and chemiluminescent conjugates (ARCHITECT® system; Abbott Laboratories, Abbott Park, Ill.; see “Bulk Reagent Random-Access Analyzer: ARCHITECT i2000” Frank A. Quinn, pages 363-367. In The Immunoassay Handbook, Second Edition, edited by David Ward, Nature Publishing Group, London, UK; U.S. Pat. No. 5,795,784 and U.S. Pat. No. 5,856,194). Assay formats examined included a 2-step format and a 1-step format.

2-Step Format

In this format, samples, specimen diluent, and coated paramagnetic microparticles were mixed into a reaction vessel, vortexed, and incubated for 18 min. Following this incubation, the microparticles were sequestered at the side of the reaction vessel using a magnet while the reaction supernatant was removed. The microparticles were subsequently washed with water/detergent solution. Antibodies present in the samples and captured on the microparticles were retained during the washing step(s). Immediately following washing, an acridinium-labelled recombinant antigen conjugate in conjugate diluent buffer, was added to the reaction vessel, which was vortexed and then allowed to incubate for 4 minutes. Incubation was followed by a second wash step and finally an activation of the acridinium and simultaneous measurement of light output, which is proportional to the amount of conjugate bound onto the microparticles.

1-Step Format

In the 1-step format, samples, coated microparticles, sample diluent, and diluted conjugate were mixed into a reaction vessel. Following an 18-minute incubation, the magnetic microparticles were captured at the side of the reaction vessel using a magnet and washed with a water/detergent mixture. Particles were then released from the vessel wall and suspended in diluent and incubated for 4 minutes. Incubation was followed by a second wash step and finally an activation of the acridinium and simultaneous measurement of light output, which is proportional to the amount of conjugate bound onto the microparticles.

Diluents

Magnetic microparticle-based chemiluminescent assays performed using the ARCHITECT® instrument utilize various diluents including sample or specimen diluent, conjugate diluent and microparticle diluent. Direct (sandwich) antibody assays for detection of HCV antibodies were developed that used various combinations of diluents provided in the ARCHITECT® commercial kits. The results of these assays are provided in the Examples below and the components of the diluents employed are listed below. Microparticles were suspended at 0.02-0.03% (w/v) solids in the respective diluent. The following abbreviations are used: SOD: superoxide dismutase; FCS: fetal calf serum; CTAB: cetyl trimethylammonium bromide; MES: 2-(N-morpholino)ethanesulfonic acid; EDTA: ethylenediaminetetraacetic acid; DTT: dithiothreitol; BSA: bovine serum albumin; NFDM: non-fat dry milk. MAK33 is an aggregated mouse monoclonal antibody (Roche Applied Science Indianapolis, Ind.). All % are weight/weight except when noted otherwise.

Diluent A

20 mM Tris, pH 7.5

0.3 M NaCl

0.05 M 6-ACA

0.15% (v/v) E. coli Lysate

75 mg/L SOD

10% (v/v) FCS

50 mg/L MAK33

5% (w/v) Triton X-405

5% (w/v) Tween-20

2.5% CTAB

2.5% SB3-14

Diluent B

20 mM MES, pH 6.6

0.14 M NaCl

5 mM EDTA

0.08% DTT

0.15% Glutathione

13.6% Sucrose

0.1% Nipasept

0.005 g/L Quinolone

Diluent C

18 mM Phosphate, pH 6.3

0.145 M NaCl

8 mM EDTA

0.1% Dextran Sulphate

0.2% non-fat dry milk

500 mg/L Poly-BSA

0.5% (v/v) FCS

1.0% (w/v) Triton X-100

0.5 gm/L Celquat

Diluent D

50 mM Tris, pH 8.4

20 mM EDTA

1.0% (w/v) Triton X-100

2.0% (v/v) Mouse Serum

0.011% Poly-L-Lysine

0.2 mg/ml Mouse IgG

0.1% Sodium azide

Diluent E

50 mM Phosphate, pH 6.3

0.5% (w/v) Triton X-405

0.1% Dextran sulphate

0.3% Poly-BSA

0.01% Mouse IgG

0.5% NFDM

0.5% BSA

5 mM EDTA 0.1% Sodium azide

### EXAMPLE 2

### Direct-Labelling of HCV NS3/Core Chimeric Protein with Acridinium

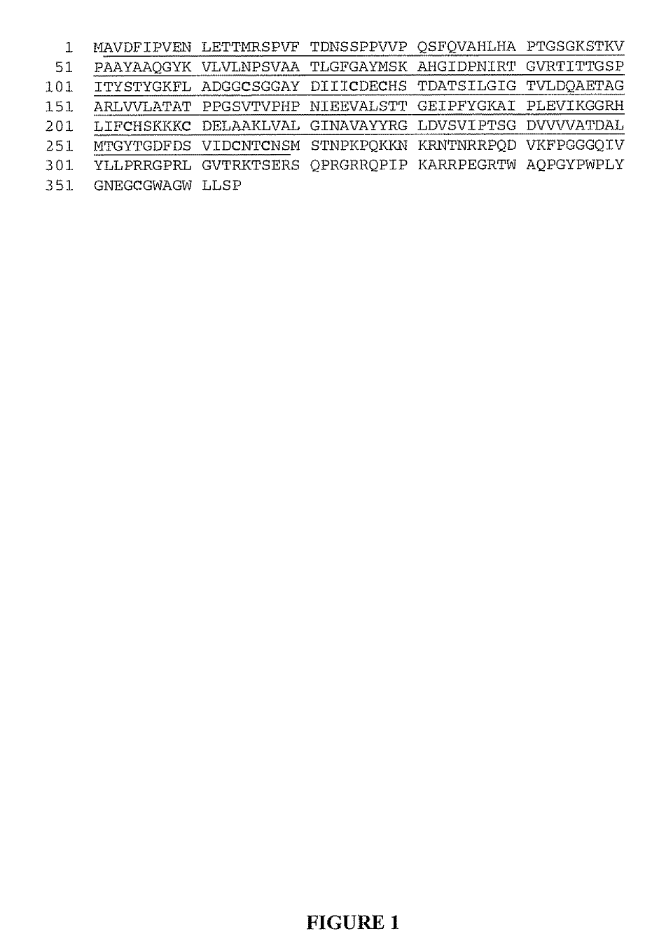

A recombinant antigen designated 9MB31 (see U.S. Pat. No. 6,855,809; also see FIG. 1 and SEQ ID NO:1), consisting of a portion of the NS3 region of HCV-1 (amino acids 1192-1457) fused, at its carboxyl-end, with the first 150 amino acids of the core protein was utilized as the binding member for detection of NS3 antibodies, 9MB31 was dialysed overnight at room temperature in PBS/SDS buffer (10 mM sodium phosphate, 150 mM sodium chloride (PBS), 0.1% sodium dodecylsulphate (SDS)) to remove DTT included in the protein buffer during purification. Dialysed protein (0.87 mg) was combined with SPSP-acridinium active ester in DMF (N,N-dimethylformamide) at various input molar ratios. Final volume of all reactions was adjusted to 0.123 mL, using PBS/SDS and reactions were incubated for 60 minutes at room temperature in the dark. Reactions were then dialysed overnight at room temperature against PBS/0.01% SDS. Dialysed, acridinylated 9MB31 was removed from the dialysis cassette and the protein concentration and acridinium incorporation determined from the absorbance at 280 nm and 370 nm by using a 1:30 dilution of the dialysate in PBS/0.01% SDS.

Conjugates prepared as described above were tested for their ability to detect antibodies against NS3 using a microparticle-based chemiluminescent immunoassay in which unmodified 9MB31 or SOD-C200 (NS3/NS4) was coated onto magnetic microparticles for capture of antibodies. Positive control samples consisted of a first pool of human plasma known to contain antibodies to the core and NS3/4 regions (PC1) and a second pool of human plasma known to contain only antibodies against the NS3 region (PC2). PC1 was used at 1:10, 1:100, and/or 1:1000 dilutions in normal (i.e. negative for HCV antibodies) human plasma, PC2 was used undiluted. The relative sensitivity of the direct-labelled 9MB31 conjugates to detect anti-NS3 antibodies is shown in Table 1 below. All conjugates were prepared using a molar ratio of 1.44 of acridinium to 9MB31.

TABLE 1
Relative Sensitivity of Directly-Labelled 9MB31 Conjugates
in Detecting NS3 Antibodies

| | | | | Signal-to-noise | |
|---|---|---|---|---|---|
| | | | | (S/N) ratios | |
| Conjugate | Antigen on | Sample | Conjugate | PC1 | |
| Concentration | Microparticle | Diluent¹ | Diluent¹ | (1:10) | PC2 |
| 10 ng/mL | 9MB31 | Kit | C | 1.6 | 1.0 |
| 50 ng/mL | 9MB31 | A | C | 6.0 | nd |
| 50 ng/mL | 9MB31 | D | E | 28.5 | 1.1 |
| 50 ng/mL | SOD-C200 | A | E | 18.0 | 1.2 |
| 50 ng/mL | SOD-C200 | kit | E | 41.0 | 1.0 |
| 200 ng/mL | SOD-C200 | D | E | 130.0 | 1.2 |

¹See Example 1 for constituents of Diluents A, C, D and E. “Kit” refers to the specimen diluent in the ARCHITECT ® HCV commercial kit (see Echevarría JM, Avellón A, Jonas G, Hausmann M, Vockel A, Kapprell HP J Clin Virol 2006(4): 368-72).

The results above indicate that with the directly labelled conjugates, signal-to-noise ratio is dependent on conjugate concentration, diluent choice and the selection of antigen coated onto the microparticle.

### Example 3

### Preparation of (Acridinium)x-Bovine Serum Albumin (Acr-BSA)

A 30% solution (300 mg/mL) of bovine serum albumin (BSA) containing 0.1% sodium azide as preservative was purchased from a commercial source (Celliance, Norcross, Ga.). Twenty mg (0.066 mL) of BSA was added to an amber glass vial containing 1.88 ML of PBS pH 7.2. To this mixture was added 5.3 mg (0.330 mL) of SPSP-acridinium active ester in DMF [N,N-dimethylformamide] (molar ratio of BSA to SPSP-acridinium active ester was 1:20). The reaction vial was capped, the solution was mixed by vortexing, and then placed at room temperature for 30-90 minutes. After incubation, the reaction volume was diluted to 5.0 mL using PBS pH 7.2. The entire diluted volume was applied to a PD-10 desalting column (GE Healthsciences) which had been equilibrated with PBS pH 7.2, 1 mM EDTA to remove unincorporated acridinium ester. The Acr-BSA was eluted from the column by using 2.2 mL of PBS pH 7.2, 1 mM EDTA. The PD-10 column eluate was then concentrated approximately 10-fold by using a Centricon YM-10 centrifugal concentrator (Amicon Corp.). The absorbance of a 30-fold dilution of concentrated Acr-BSA in PBS was measured at 280 and 370 nm for the estimation of protein concentration and calculation of incorporated acridinium per BSA molecule. The concentration was calculated 4.0 mg/ml with 9.0 acridiniums per BSA molecule

### Example 4

### Attaching Maleimide Group to Acr-BSA

Nine hundred micrograms (0.702 mL) of Acr-BSA (from Example 3) was added to an amber glass vial containing 0.140 mL of 0.2M sodium phosphate, pH 9.0 to which was added 0.0238 mL of Succinimidyl 4-(N maleimidomethyl)-cyclohexane-1-carboxy-6-amidocaproate (LC-SMCC, Pierce Chemical Co., Rockford, Ill.) which was freshly dissolved in DMF to 5 mM. The reaction vial was capped and the solution mixed by vortexing gently and then placed at room temperature for 60 minutes in the dark. The reaction mixture was then desalted to remove unincorporated LC-SMCC by applying to a PD-10 column pre-equilibrated with PBS pH 7.2, 1 mM EDTA. LC-SMCC activated Acr-BSA (Acr-BSA-LC-Mal) was eluted with PBS pH 7.2, 1 mM EDTA. The yellow peak containing the Acr-BSA-LC-Mal at the elution solvent front was collected and concentrated approximately 5-fold by using a Centricon YM-10 centrifugal concentration device. The absorbance of a 30-fold dilution of the material in PBS was measured at 280 and 370 nm for the estimation of protein concentration. The protein concentration was calculated at 8.4 mg/ml. The Acr-BSA-LC-Mal was stored in a glass amber vial at 2-8° C.

### Example 5

### Conjugation of Reduced 9MB31 Recombinant Antigen to Acr-BSA-LC-Mal

To conjugate 9MB31 recombinant antigen to the Acr-BSA-LC-Mal (from Example 4), 9MB31 was first reduced with dithiothreitol (DTT) as follows: 2.00 mg of 9MB31 was incubated for 60 min at room temperature in the presence of 45 mM DTT in PBS pH 7.2 containing 5 mM EDTA at a final protein concentration of 2.50 mg/mL. Following reduction, the reaction mixture was applied to a PD-10 desalting column (GE Healthsciences) equilibrated in PBS pH 7.2, 0.1% SDS and eluted with 2.2 mL of equilibration buffer. The entire eluate (2.20 mL, referred to as 9MB31-SH) was applied to a Centricon YM-10 centrifugal concentration device (Amicon Corp.) and concentrated about 4-fold via centrifugation. The protein concentration of the retentate was determined by measuring the absorbance at 280 nm of a 30-fold dilution in PBS/SDS buffer. The final concentration of reduced 9MB31 was 1.107 mg/mL and total yield was 0.664 mg protein. The 9MB31-SH was then mixed at various molar ratios with Acr-BSA-LC-Mal (50 micrograms per reaction; molar ratios based on the molecular weight of Acr-BSA-LC-Mal) in a final reaction volume of 0.200 mL and incubated overnight at room temperature in the dark. Conjugates were then diluted to 250 ng/mL in Diluent E (see Example 1) for testing.

### Example 6

### Relative Performance of Acr-BSA-LC-9MB31 Conjugates

The ability of the Acr-BSA-LC-9MB31 conjugates described in Example 5 to detect HCV NS3 antibodies was assessed using the ARCHITECT® instrument in a 2-step format as described in Example 1. Diluent D was selected for use as the sample diluent based on previous experiments indicating superior results utilising this diluent compared to Diluent A. Two types of magnetic microparticles were examined, those coated with 9MB31 recombinant antigen (untreated) and those coated with SOD-C200 recombinant antigen. The results are shown in Table 2 and demonstrate that the indirectly-labelled conjugates outperformed the directly labelled 9MB31 conjugates (cf. data in Table 1 (Example 2) at tows 3 and 6) for detection of PC1 diluted 1:10 in normal human plasma. SOD-C200 coated microparticles outperformed 9MB31 coated microparticles. PC2 reactivity was highest with the indirectly labelled conjugate prepared with a 1:0.5 to 1:2 ratio of Acr-BSA-LC-Mal to 9MB31-SH depending on the recombinant antigen coated microparticles used.

TABLE 2
Relative Performance of Indirectly Labelled
Acr-BSA-LC-9MB31 Conjugates

| | | Signal-to-negative (S/N) ratios | | | |
|---|---|---|---|---|---|
| | | 9MB31 coated | | SOD-C200 coated | |
| Conjugate | Molar ratio | microparticles | | microparticles | |
| Number | Acr-BSA:9MB31 | PC1 (1:10) | PC2 | PC1 (1:10) | PC2 |
| 1 | 1:0.125 | 13.05 | 1.31 | 281.96 | 1.91 |
| 2 | 1:0.25 | 110.61 | 1.81 | 223.67 | 2.38 |
| 3 | 1:0.5 | 81.44 | 1.98 | 213.68 | 5.47 |
| 4 | 1:1 | 90.47 | 1.91 | 240.60 | 2.14 |
| 5 | 1:2 | 92.92 | 2.13 | 284.54 | 2.10 |
| 6 | 1:3 | 83.91 | 1.79 | 280.57 | 2.13 |
| 7 | 1:4 | 79.65 | 1.79 | 308.49 | 2.24 |

The results above show that assay signal-to-noise ratio for PC1 is highest when SOD-C200 coated paramagnetic microparticles are used, and for PC2 detection, when Acr-BSA-LC-Mal:9MB31 molar ratio is 1:0.5. Compared to directly labelled 9MB31 (see Example 2), the indirectly labelled conjugate provided higher signal-to-noise values for both PC1 and PC2.

### Example 7

### Preparation of Acr-BSA-LC-9MB31 Conjugate Following Urea Dialysis of 9MB31 Recombinant Antigen

Prior to reduction and conjugation with Acr-BSA-LC-Mal, 9MB31 recombinant antigen was dialysed against 50 mM HEPES pH 6.8, 1 mM EDTA (HEPED) containing 2M, 4M or 8M urea. Following dialysis, the protein concentration of the dialysate was calculated from the absorbance at 280 nm of a 1:30 dilution in PBS pH 7.2. Urea-dialysed protein was reduced with DDT as described in Example 5 except that HEPED buffers containing 2M, 4M, or 8M urea were used. Following reduction, excess DDT was removed by using centrifugal desalting column (Zeba Spin Column, Pierce Chemical Co.) and the protein concentration of the recovered protein determined from the absorbance at 280 nm of a 1:30 dilution in PBS.

As recovery from the desalting column in this experiment was low for 9MB31-SH in 2M or 4M urea, only 8M urea dialysed/reduced 9MB31 was used for conjugation to Acr-BSA-LC-Mal. The BSA:9MB31 molar ratios used for the 15 conjugation reactions conducted are shown in Table 3 below. The amount of Acr-BSA-LC-Mal was held constant in all reactions. Reactions 1-5 used HEPED buffer for diluting the reaction mixture to the final volume of 0.200 mL, while reactions 6-10 used HEPED containing 8M urea and reactions 11-15 were undiluted. The reactions were incubated overnight in the dark.

TABLE 3
Reaction Conditions for Preparation of Urea-Dialysed,
Indirectly Labelled Acr-BSA-LC-9MB31 Conjugates

| | | | | Final | |
|---|---|---|---|---|---|
| | | | | SPSP- | Final |
| | | | Final | BSA | 9MB31 |
| | Molar ratio | | Volume | conc | conc |
| Rxn | BSA:9MB31 | Buffer Added | (mL) | (mg/mL) | (mg/mL) |
| 1 | 4:1 | HEPED | 0.20 | 0.250 | 0.033 |
| 2 | 2:1 | HEPED | 0.20 | 0.250 | 0.067 |
| 3 | 1:1 | HEPED | 0.20 | 0.250 | 0.134 |
| 4 | 1:2 | HEPED | 0.20 | 0.250 | 0.267 |
| 5 | 1:4 | HEPED | 0.20 | 0.250 | 0.535 |
| 6 | 4:1 | HEPED, 8M urea | 0.20 | 0.250 | 0.033 |
| 7 | 2:1 | HEPED, 8M urea | 0.20 | 0.250 | 0.067 |
| 8 | 1:1 | HEPED, 8M urea | 0.20 | 0.250 | 0.134 |
| 9 | 1:2 | HEPED, 8M urea | 0.20 | 0.250 | 0.267 |
| 10 | 1:4 | HEPED, 8M urea | 0.20 | 0.250 | 0.535 |
| 11 | 4:1 | none | 0.02 | 2.868 | 0.383 |
| 12 | 2:1 | none | 0.02 | 2.646 | 0.708 |
| 13 | 1:1 | none | 0.02 | 2.292 | 1.226 |
| 14 | 1:2 | none | 0.03 | 1.808 | 1.934 |
| 15 | 1:4 | none | 0.04 | 1.271 | 2.720 |

The conjugates were diluted to 250 ng/mL in Diluent E for testing in the 2-step ARCHITECT® assay as described in Example 1. Diluent D was used as the specimen diluent. SOD-C200 coated microparticles were also used. The results are presented in Table 4 and show that the signal (S/N) values increased with increasing amounts of urea-dialysed 9MB31-SH in the reactions. Those conjugates prepared in the presence of the highest urea concentration (reactions 6-10) had the lowest S/N values while those in which urea was diluted to a final concentration of 0.06M-0.93M, corresponding to 0.7%-11.7% of the initial concentration (i.e. reactions 1-5) had the highest S/N values Compared to the Acr-BSA-LC-9MB31 conjugate prepared without prior urea dialysis (control conjugate 3, from Example 7 see row 1 of Table 4), the conjugates prepared from the urea-dialysed conjugates exhibited 200-250% higher SIN values for PC1 and PC2.

TABLE 4
Relative Performance of the Urea-Dialysed, Indirectly
Labelled Acr-BSA-LC-9MB31 Conjugates
in a 2-Step Assay Format

| | | | Mean | S/N | |
|---|---|---|---|---|---|
| | Molar ratio | | background | PC1 | S/N |
| Rxn | BSA:9MB31 | Buffer added | RLU | (1:10) | PC2 |
| Conj 3, | 1:2 | na | 484.4 | 181.1 | 3.4 |
| Example 7 | | | | | |
| 1 | 4:1 | HEPED | 378.8 | 37.9 | 1.3 |
| 2 | 2:1 | HEPED | 311.2 | 70.3 | 1.9 |
| 3 | 1:1 | HEPED | 470.2 | 109.4 | 2.2 |
| 4 | 1:2 | HEPED | 365.8 | 389.5 | 6.5 |
| 5 | 1:4 | HEPED | 452.0 | 455.1 | 6.8 |
| 6 | 4:1 | HEPED, 8M | 290.4 | 6.9 | 0.7 |
| | | urea | | | |
| 7 | 2:1 | HEPED, 8M | 339.8 | 12.3 | 0.9 |
| | | urea | | | |
| 8 | 1:1 | HEPED, 8M | 245.4 | 40.5 | 2.0 |
| | | urea | | | |
| 9 | 1:2 | HEPED, 8M | 462.2 | 61.2 | 1.7 |
| | | urea | | | |
| 10 | 1:4 | HEPED, 8M | 497.8 | 148.5 | 3.5 |
| | | urea | | | |
| 11 | 4:1 | none | 347.6 | 30.9 | 1.7 |
| 12 | 2:1 | none | 445.0 | 55.8 | 1.6 |
| 13 | 1:1 | none | 423.0 | 150.7 | 3.2 |
| 14 | 1:2 | none | 581.8 | 180.6 | 4.4 |
| 15 | 1:4 | none | 836.2 | 223.1 | 4.2 |

The results in Table 4 show that assay signal-to-noise ratio is highest for PC2 when using a molar ratio of Acr-BSA-LC-Mal:9MB31 of 1:2 to 1:4 and when the buffer added to the reaction mixture contains no urea. Compared to directly labelled conjugate (see Example 2) or indirectly labelled conjugate that comprises non-urea dialysed 9MB31 antigen (see Example 6), the indirectly-labelled conjugate incorporating the urea-dialysed antigen is more potent.

### Example 8

### Evaluation of Acr-BSA-LC-9MB31 (Urea-Dialysed) Conjugates in a 1-Step Vs. a 2-Step Immunoassay Format 31

The “Reaction 5” conjugate from Example 7 (see Tables 3 and 4) was diluted to 250 ng/mL in Diluent D or Diluent E and used in either 1-step or 2-step immunoassays on the ARCHITECT® instrument as described in Example 1. SOD-C200 coated microparticles were used. The results are presented in Table 5 below. As shown in Table 5, utilization of Diluent E as both the specimen diluent and the conjugate diluent in the 1-step format significantly improved the S/N values obtained for PC1 (used at 1:100 dilution in normal human plasma rather than 1:10 as in the previous Examples), as well as that of PC2 and positive control 3 (PC3). PC3 is an undiluted human serum from an HCV infected individual who possesses only antibodies directed against HCV NS3 encoded protein

TABLE 5
Relative Performance of Reaction 5 Acr-BSA-LC-9MB31
(Urea-Dialysed) Conjugate in 1-Step and 2-Step Assay Formats

| Assay | Specimen | Conjugate | S/N values | | |
|---|---|---|---|---|---|
| Format | Diluent | Diluent | PC1 (1:100) | PC2 | PC3 |
| 2-step | D | E | 47.7 | 2.9 | 2.4 |
| 1-step | D | E | 22.6 | nd | nd |
| 1-step | D | D | 25.0 | 3.2 | nd |
| 1-step | E | E | 292.4 | 20.6 | 123.9 |

The results in Table 5 show that the one-step assay format provides superior signal-to-noise ratios for both PC2 and PC3 (which are both human specimens known to contain only anti-NS3 antibodies) when using indirectly-labelled 9MB31 wherein 9MB31 had been urea-dialysed prior to conjugation to Acr-BSA-LC-Mal. Choice of diluent composition has an effect on assay sensitivity.

### Example 9

### Effect of Diluent pH and Composition on Relative Sensitivity of Acr-BSA-LC-9MB31 (Urea-Dialysed) Conjugate

The results shown in Table 5 above suggest that the diluent used in the 1-step assay can influence the sensitivity of anti-NS3 detection. Thus, other diluent combinations or modifications of diluents were examined in a 1-step antibody sandwich assay using SOD-C200 coated magnetic microparticles and the Acr-BSA-LC-9MB31 conjugate prepared as described in Example 7, Reaction 4 (1:2 ratio of Acr-BSA-LC-Mal to 9MB31-SH). The results are presented in Table 6 below and demonstrate that using this conjugate, the S/N values can be increased for PC1, PC2 and PC3 by employing Diluent E with an increased pH as the specimen diluent, or by substituting Diluent C for Diluent E as the specimen diluent.

TABLE 6
Relative Performance of Reaction 4 Acr-BSA-LC-9MB31
(Urea-Dialysed) Conjugate using Various Specimen Diluents

| Assay | Specimen | Conjugate | S/N values | | |
|---|---|---|---|---|---|
| Format | Diluent | Diluent | PC1 (1:100) | PC2 | PC3 |
| 1-step | E | E | 291.0 | 39.9 | 107.2 |
| 1-step | E, pH 8.4 | E | 309.1 | 63.8 | 205.4 |
| 1-step | C | E | 361.7 | 55.0 | 266.1 |

The results in Table 6 shown that diluent composition and/or pH affects signal-to-noise values in the one-step assay format utilising indirectly-labelled 9MB33 wherein 9MB31 had been urea-dialysed prior to conjugation to Acr-BSA-LC-Mal.

### Example 10

### Conjugation and Performance in a 1-Step Immunoassay of Other HCV NS3-Derived Recombinant Antigens Following Urea Dialysis

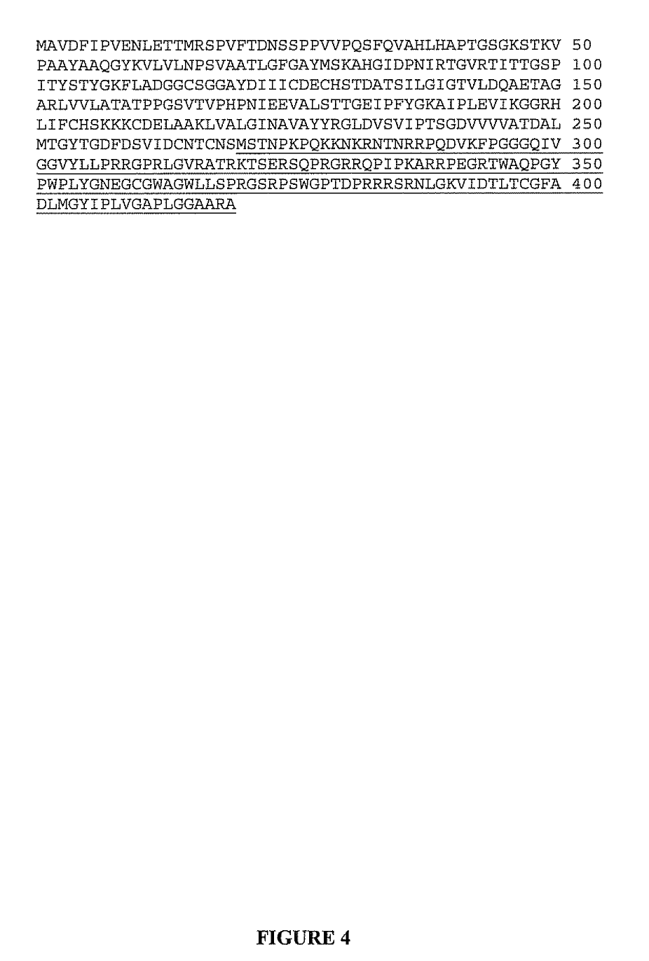

Indirectly labelled conjugates comprising other NS3-derived recombinant antigens have been successfully prepared using the method described in Example 8, which comprises the step of dialysing the antigen in urea prior to conjugation to Acr-BSA-LC-Mal. The resulting conjugates were used successfully in an HCV anti-NS3 sandwich antibody assay. Specifically, Acr-BSA-LC-antigen conjugates were prepared as described in Example 8 by conjugating, in addition to 9MB31, the following HCV recombinant antigens to Acr-BSA-LC-Mal:
HC31 (an E. coli expressed recombinant antigen comprising amino acids 1192-1457 of NS3 fused at the C-terminus to amino acids 1676-1931 of NS4; see U.S. Pat. No. 5,312,737);
SOD-C200 (a yeast expressed recombinant antigen comprising amino acids 1192-1931 of NS3; see U.S. Pat. No. 5,350,671), and
HC43 (an E. coli expressed recombinant antigen comprising amino acids 1192-1457 of NS3 fused at the C-terminus to amino acids 1-150 of the core protein; SEQ ID NO:2 (see FIG. 4 and U.S. Pat. No. 5,705,330).

The above conjugates were diluted to 250 ng/mL in Diluent E and utilized in a 1-step anti-NS3 sandwich assay using SOD-C200 coated microparticles as described in Example 1. Diluent E was used as the specimen diluent. The results, shown in Table 7, demonstrate that each of the four Acr-BSA-LC conjugates were able to detect a 1:100 dilution of PC1 plasma with S/N values greater than 42.0.

TABLE 7
Relative Performance of Various Urea-Dialysed,
Indirectly Labelled NS3 Antigen Conjugates

| Recombinant | | Mean | | | |
|---|---|---|---|---|---|
| Antigen | Molar ratio | Background | S/N values | | |
| (rAg) | Acr-BSA:rAg | RLU | PC1 (1:100) | PC2 | PC3 |
| 9MB31 | 1:2 | 1054.3 | 238.1 | 12.8 | 141.0 |
| 9MB31 | 1:4 | 1258.5 | 229.6 | 10.2 | 19.4 |
| C200 | 1:2 | 1254.0 | 43.9 | nd | nd |
| C200 | 1:4 | 1218.8 | 50.1 | nd | nd |
| HC43 | 1:2 | 1483.8 | 71.6 | 9.4 | 172.8 |
| HC43 | 1:4 | 184.8 | 42.4 | 1.9 | 2.6 |
| HC31 | 1:4 | 1218.8 | 53.9 | nd | nd |

The results shown in Table 7 show that Acr-BSA-LC-Conjugates comprised of urea-dialysed HCV NS3-derived antigens other than 9MB31 can readily be synthesised and are capable of detecting cognate antibodies with good sensitivity in the 1-step sandwich assay format.

### Example 11

### Comparison of Acr-BSA-LC-rAg Conjugates Prepared With and Without Prior Urea Dialysis of the Recombinant Antigen

Conjugates were prepared using the recombinant antigens 9MB31 and HC43 by the methods described in Examples 5 and 7. The resulting conjugates were then diluted to 250 ng/mL in Diluent E and utilized in a 1-step anti-NS3 sandwich assay using SOD-C200 coated microparticles as described in Example 1. Diluent E was used as the specimen diluent.

The results, as shown in Table 8, demonstrate that Acr-BSA-LC-antigen conjugates made with recombinant antigens that were submitted to a urea dialysis step prior to conjugation exhibited higher S/N values at all PC1 dilutions as compared to conjugates made with non-urea dialysed recombinant antigen.

These results indicate that the use of urea dialysis is capable of increasing the sensitivity of conjugates employing a variety of antigens.

TABLE 8
Relative Performance of Urea-Dialysed and Non-Urea
Dialysed Indirectly Labelled Conjugates using PC1

| Recombinant | | | S/N Values | | | |
|---|---|---|---|---|---|---|
| Antigen | Molar Ratio | Mean Background | 1:10 | 1:100 | 1:1000 | 1:2000 |
| (rAg) | BSA:rAg | RLU | Dilution | Dilution | Dilution | Dilution |
| 9MB31 | 1:2 | 1400.8 | 98.6 | 120.6 | 25.4 | 13.5 |
| w/o urea | | | | | | |
| 9MB31 + urea | 1:2 | 1185.5 | 155.0 | 200.9 | 41.8 | 21.0 |
| HC43 | 1:2 | 921.8 | 33.8 | 38.5 | 9.5 | 5.4 |
| w/o urea | | | | | | |
| HC43 + urea | 1:2 | 1483.8 | 100.7 | 71.2 | 15.5 | 7.9 |

The results in Table 8 show that urea dialysis of HCV NS3 recombinant antigens prior to conjugation to Acr-BSA-LC-Mal produces more potent conjugates than those synthesized with antigens that have not been dialysed in urea-containing buffer prior to conjugation.

### Example 12

### Performance Comparison of Directly Labelled, Indirectly Labelled and Urea-Dialysed, Indirectly Labelled Conjugates

Results from Examples 2 and 6 to 11 are summarised in Table 9 (2-step format) and Table 10 (1-step format) below. Only results using SOD-C200 coated microparticles are shown as these gave superior results as compared to 9MB31 coated microparticles.

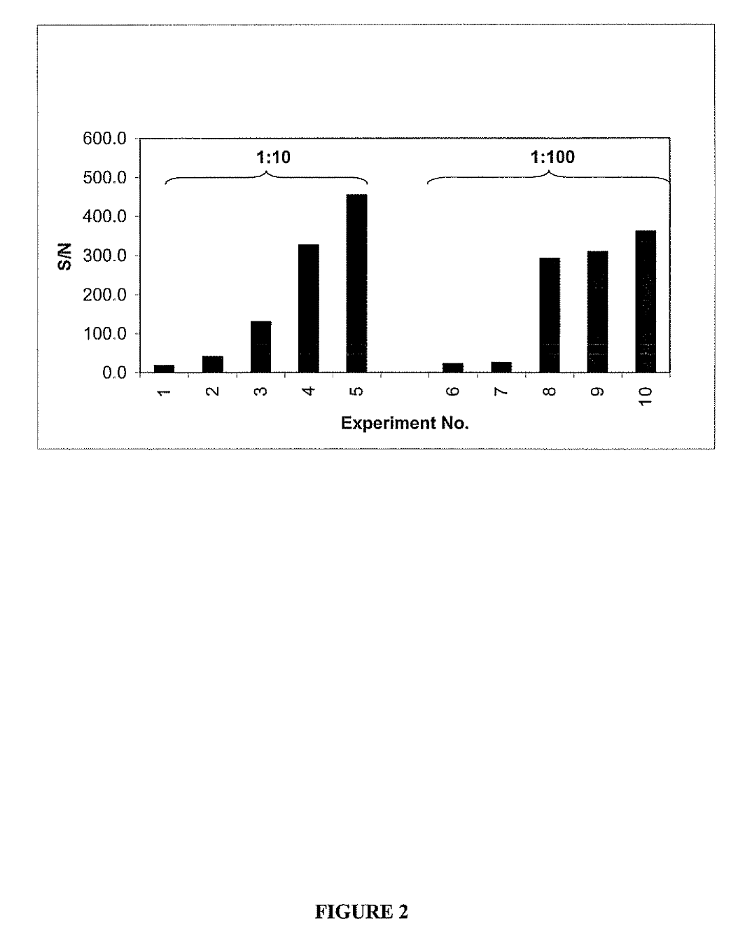

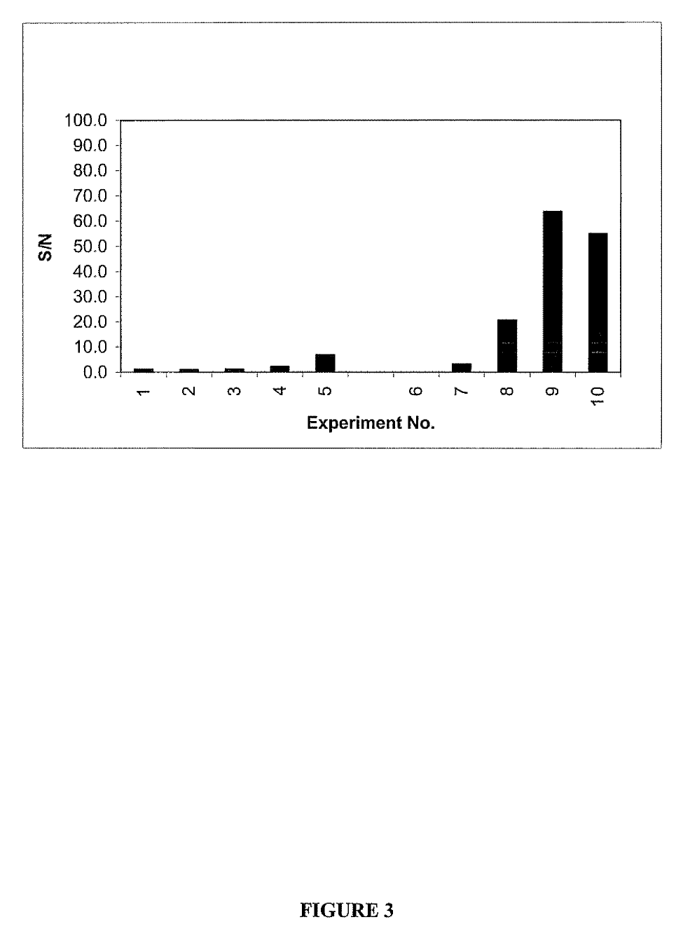

As can be seen from Table 9 and FIGS. 2 and 3, the use of a urea-dialysed, indirectly labelled conjugate significantly increases the sensitivity of the assay. Specifically, the results indicate that modifications to the conjugation process or assay parameters produced a more potent conjugate and a more robust immunoassay. For example, progressing from directly labelled, to indirectly labelled, to indirect labelling of urea-dialysed antigens leads to an 18-fold increase in assay signal-to-noise ratio for PC1 and an approximately 3-fold increase in signal-to-noise value for PC2 (Table 9). Use of a 1-step assay format and diluent selection (or pH) can also affect the sensitivity of the assay (Table 10).

TABLE 9
Comparison of Performance of Various Conjugates in
Detection of NS3 Antibodies in a 2-Step Assay Format

| | | S/N Values | | | | |
|---|---|---|---|---|---|---|
| | | PC1 | | Parameter leading to | Sample | Conjugate |
| Expt # | Conjugate | (1:10) | PC2 | Improvement | Diluent | Diluent |
| 1 | Acr-9MB31 | 18.0 | 1.2 | Purify Acr-9MB31 via | A | E |
| | | | | dialysis in 0.01% SDS | | |
| 2 | Acr-9MB31 | 41.0 | 1.0 | Purify Acr-9MB31 via | Kit | E |
| | | | | dialysis in 0.01% SDS, | | |
| | | | | C200 mps, diluent | | |
| 3 | Acr-9MB31 | 130.1 | 1.2 | Sample diluent | D | E |
| | | | | substitution, increase | | |
| | | | | conjugate concentration | | |
| | | | | 4x | | |
| 4 | Acr-BSA- | 326.0 | 2.2 | Indirect labelling method | D | E |
| | 9MB31 | | | | | |
| 5 | Acr-BSA- | 455.1 | 6.8 | Indirect labelling method | D | E |
| | (urea)9MB31 | | | using urea-dialysed | | |
| | | | | antigen | | |

TABLE 10
Comparison of Performance of Various Conjugates in Detection of NS3
Antibodies in a 1-Step Assay Format

| | | S/N Values | | | | Parameter | | |
|---|---|---|---|---|---|---|---|---|
| | | PC1 | PC1 | | | leading to | Sample | Conjugate |
| Expt # | Conjugate | (1:10) | (1:100) | PC2 | PC3 | Improvement | Diluent | Diluent |
| 6 | Acr-BSA- | 22.9 | 22.6 | nd | nd | Indirect labelling | D | E |
| | (urea)9MB31 | | | | | method using | | |
| | | | | | | urea-dialysed | | |
| | | | | | | antigen | | |
| 7 | Acr-BSA- | 21.5 | 25.0 | 3.2 | nd | Indirect labelling | D | D |
| | (urea)9MB31 | | | | | method using | | |
| | | | | | | urea-dialysed | | |
| | | | | | | antigen, substitute | | |
| | | | | | | diluent D for E as | | |
| | | | | | | conjugate diluent | | |
| 8 | Acr-BSA- | 288.9 | 292.4 | 20.6 | 123.9 | 1-step assay | E | E |
| | (urea)9MB31 | | | | | format, indirect | | |
| | | | | | | labelling method | | |
| | | | | | | using urea- | | |
| | | | | | | dialysed antigen, | | |
| | | | | | | substitute diluent | | |
| | | | | | | E for D as sample | | |
| | | | | | | diluent | | |
| 9 | Acr-BSA- | 254.3 | 309.1 | 63.8 | 205.4 | Indirect labelling | E @ | E |
| | (urea)9MB31 | | | | | method using | pH 8.4 | |
| | | | | | | urea-dialysed | | |
| | | | | | | antigen, diluent E | | |
| | | | | | | at pH 8.4 as | | |
| | | | | | | sample diluent | | |
| 10 | Acr-BSA- | 271.6 | 361.7 | 55.0 | 266.1 | Indirect labelling | C | E |
| | (urea)9MB31 | | | | | method using | | |
| | | | | | | urea-dialysed | | |
| | | | | | | antigen, diluent C | | |
| | | | | | | as sample diluent | | |

### Example 13

### Analytical Sensitivity of Acr-BSA-LC-9MB31 (Urea-Dialysed) Conjugate in a 1-Step Assay Format

To determine the relative sensitivity of the Acr-BSA-LC-9MB31 (urea-dialysed) conjugate in a 1-step anti-HCV NS3 assay with that of the ARCHITECT® anti-HCV commercial kit (Abbott List No. 6C37), serial dilutions of PC1 (in normal human plasma) were tested. The commercial ARCHITECT® anti-HCV assay kit was used as described in the package insert. The assay protocol described in the package insert is a 2-step, indirect format in which conjugates directed against human IgG and IgM are used to detect antibodies bound to HCV core and NS3/NS4 recombinant antigens. The 1-step assay using Acr-BSA-LC-9MB31 (urea dialysed) conjugate will only detect antibodies directed against the NS3 region shared between SOD-C200 (solid phase antibody capture reagent) and 9MB31 used in the conjugate. Diluent E was used both as the specimen diluent and for dilution of conjugate (final conjugate concentration: 250 ng/mL) in the 1-step assay using the Acr-BSA-LC-9MB31 (urea dialysed) conjugate.

The results of the comparison are shown in Table 11 below and indicate that the 1-step anti-NS3 sandwich assay employing the Acr-BSA-LC-9MB31 (urea-dialysed) conjugate exhibits S/N values for the PC1 dilution panel that are greater than that of the commercial antibody kit even without the benefit of anti-core or anti-NS4 IgG and IgM detection of the commercial ARCHITECT® anti-HCV assay.

TABLE 11
Comparison of a 1-Step Assay Utilising the Acr-BSA-LC-9MB31
(Urea-Dialysed) Conjugate with the Commercial
ARCHITECT ® Anti-HCV Kit using PC1

| | S/N Values | | |
|---|---|---|---|
| Assay | 1:100 Dilution | 1:1000 Dilution | 1:2000 Dilution |
| ARCHITECT ® | 77.0 | 19.1 | 10.7 |
| Anti-HCV Kit | | | |
| 1-Step Anti-NS3 | 200.9 | 41.8 | 21.0 |
| Assay utilising | | | |
| Urea-Dialysed | | | |
| 9MB31 Conjugate | | | |

The results in Table 11 show that analytical sensitivity of the 1-step anti-HCV NS3 sandwich immunoassay using urea dialysed, indirectly labelled 9MB31 is superior to the ARCHITECT® anti-HCV kit even though the kit reagents allow detection of NS3, core and NS4 antibodies using an indirect assay format (i.e. anti-human conjugate)

### Example 14

### Testing of Commercial HCV Seroconversion Panels

Seroconversion panels purchased from commercial vendors (ZeptoMetrix (Buffalo, N.Y.), Boston Biomedica, Inc. (BBI, West Bridgewater, Mass.), and North America Biological, Inc., (NABI, Boca Raton, Fla.) were tested with the Commercial ARCHITECT® anti-HCV assay kit (Abbott List No. 6C37) per package insert instructions. Panels were also tested by using the 1-step anti-NS3 sandwich assay as described in Example 13.

The results are shown in Table 12. The confirmatory HCV RIBA-3 immunoblot assay results were provided by the vendors of the seroconversion panels. The panels are designated as possessing anti-core and/or anti-NS3 antibodies based on RIBA-3 results. The 1-step anti-NS3 sandwich assay was predicted not to detect antibodies in any of the panels designated as “anti-core” since RIBA-3 did not detect NS3 antibodies in these panels. However, very high S/N values were obtained for all members of Panel PHV912. Among those panels possessing NS3 antibodies, it can be seen that employing the indirectly-labelled, urea dialysed conjugates and the 1-step assay generally showed superior results to the commercial anti-HCV assay kit. The lower sensitivity in some cases is likely due to the fact that the sandwich assay presents a limited repertoire of the NS3 epitopes in a favourable fashion but, owing to the greater signal generating capacity of the indirectly-labelled, urea-dialysed conjugate, antibodies reactive to these epitopes are more readily detected by this conjugate than by the commercial kit. In addition, the immune response is likely to be variable within the population as some individuals produce antibodies against certain epitopes earlier (or later) during infection than others and with higher (or lower) titers. Thus, the sandwich assay can only detect antibodies against epitopes that are presented in the proper conformation,

TABLE 12
Comparison of Detection of NS3 Antibodies in Commercial HCV Seroconversion
Panels using ARCHITECT ® Anti-HCV Commercial Kit or the 1-Step
Anti-NS3 Sandwich Assay

| | Anti-NS3 Sandwich | ARCHITECT ® Anti- | |
|---|---|---|---|
| | Assay, 1-step | HCV Kit | |
| Seroconversion Panel | S/N | S/CO | RIBA 3.0 |
| 6215, n1 (Anti-Core) | 0.88 | 0.027 | neg |
| 6215, n2 | 0.88 | 0.026 | neg |
| 6215, n3 | 0.87 | 0.073 | neg |
| 6215, n4 | 1.3 | 2.965 | Ind (core) |
| SC-0402, n1 (Anti-Core) | 1.4 | 0.054 | neg |
| SC-0402, n2 | 1.5 | 0.039 | neg |
| SC-0402, n3 | 1.3 | 0.147 | neg |
| SC-0402, n4 | 1.1 | 1.445 | Ind (core) |
| PHV909, n1 (Anti-Core) | 1.7 | 0.095 | neg |
| PHV909, n2 | 1.7 | 1.696 | Ind (core) |
| PHV909, n3 | 1.7 | 1.836 | Ind (core) |
| PHV912, n1 (Anti-Core) | 253.7 | 0.281 | neg |
| PHV912, n2 | 182.7 | 0.226 | neg |
| PHV912, n3 | 70.6 | 10.703 | Ind (core) |
| PHV913, n1 (Anti-Core) | 0.87 | 0.086 | neg |
| PHV913, n2 | 0.78 | 0.272 | neg |
| PHV913, n3 | 1.15 | 2.868 | Ind (core) |
| PHV913, n4 | 1.0 | 2.787 | Ind (core) |
| PHV918, n1 (Anti-Core) | 0.7 | 0.058 | neg |
| PHV918, n2 | 0.78 | 0.059 | neg |
| PHV918, n3 | 0.64 | 0.072 | neg |
| PHV918, n4 | 0.7 | 0.061 | neg |
| PHV918, n5 | 0.81 | 0.086 | neg |
| PHV918, n6 | 0.73 | 0.151 | neg |
| PHV918, n7 | 1.0 | 4.122 | Ind (core) |
| PHV918, n8 | 4.1 | 3.783 | core/NS3 |
| 6212, n1 (Anti-NS3) | 1.0 | 0.033 | neg |
| 6212, n2 | 38.0 | 0.953 | neg |
| 6212, n3 | 42.1 | 1.240 | neg |
| 6212, n4 | 83.0 | 5.569 | Ind (NS3) |
| 6212, n5 | 80.3 | 6.302 | Ind (NS3) |
| 6212, n6 | 51.0 | 6.569 | Ind (NS3) |
| 6212, n7 | 34.1 | na | Ind (NS3) |
| 6212, n8 | 50.3 | na | Ind (NS3) |
| 6212, n9 | 48.5 | 10.583 | Ind (NS3) |
| 6214, n1 (Anti-NS3) | 1.3 | 0.071 | neg |
| 6214, n2 | 1.2 | 0.066 | neg |
| 6214, n3 | 1.1 | 0.058 | neg |
| 6214, n4 | 1.1 | 0.057 | neg |
| 6214, n5 | 1.0 | 0.057 | neg |
| 6214, n6 | 1.1 | 0.056 | neg |
| 6214, n7 | 2.5 | 0.218 | neg |
| 6214, n8 | 2.0 | na | neg |
| 6214, n9 | 2.8 | 3.625 | Ind (NS3) |
| 6214, n10 | 3.5 | 6.065 | Ind (NS3) |
| 6214, n11 | 27.4 | 11.382 | pos (NS3/NS4) |
| 6214, n12 | 16.5 | 11.387 | pos |
| 6214, n13 | 14.6 | 11.615 | pos |
| SC-0403, n1 (Anti-NS3) | 0.9 | 0.036 | neg |
| SC-0403, n2 | 321.3 | 13.338 | pos |
| | | | (core/NS3/4/5) |
| SC-0403, n3 | 303.2 | 13.518 | pos |
| | | | (core/NS3/4/5) |
| SC-0403, n4 | 296.7 | 13.417 | pos |
| | | | (core/NS3/4/5) |
| SC-0403, n5 | 238.9 | 12.806 | pos |
| | | | (core/NS3/4/5) |
| SC-0406, n1 (Anti-NS3) | 1.2 | 0.055 | neg |
| SC-0406, n2 | 2.2 | 2.015 | pos (core/NS3) |
| SC-0406, n3 | 2.9 | 4.375 | pos (core/NS3) |
| PHV919, n1 (Anti-NS3) | 1.0 | 0.384 | neg |
| PHV919, n2 | 1.2 | 0.463 | neg |
| PHV919, n3 | 1.0 | 0.318 | neg |
| PHV919, n4 | 1.1 | 0.579 | neg |
| PHV919, n5 | 13.3 | 5.623 | pos (core/NS3) |
| PHV919, n6 | 39.0 | 11.666 | pos (core/NS3) |
| PHV919, n7 | 33.4 | 11.779 | pos (core/NS3) |
| PHV908, n1 (Ant-NS3) | 1.01 | 0.058 | neg |
| PHV908, n2 | 0.79 | 0.062 | neg |
| PHV908, n3 | 1.03 | 0.092 | neg |
| PHV908, n4 | 2.0 | 1.404 | pos (NS3, NS4) |
| PHV908, n5 | nd | 1.478 | pos (NS3, NS4) |
| PHV908, n6 | 2.6 | 4.283 | pos (NS3, NS4) |
| PHV908, n7 | 3.1 | 8.267 | pos (NS3, NS4) |
| PHV908, n8 | 3.7 | 8.388 | pos (NS3, NS4) |
| PHV908, n9 | 7.8 | 9.964 | pos (NS3, NS4) |
| PHV908, n10 | 9.8 | 9.607 | pos (NS3, NS4) |
| PHV908, n11 | 14.5 | 10.362 | pos (NS3, NS4) |
| PHV908, n12 | 16.2 | 10.656 | pos (NS3, NS4) |
| PHV908, n13 | 17.0 | 10.889 | pos (NS3, NS4) |

Descriptions in the RIBA 3.0 results column indicate which HCV antigen, if any, was detected.
neg = negative for HCV antibodies
ind = indeterminant per RIBA-3 package insert instructions
S/N = signal-to-moise ratio
S/CO = signal-to-cutoff ratio

The disclosure of all patents, publications, including published patent applications, and database entries referenced in this specification are expressly incorporated by reference in their entirety to the same extent as if each such individual patent, publication, and database entry were expressly and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

What is claimed is:

1. A process for preparing a protein conjugate, said process comprising the steps of:
(a) subjecting a protein to denaturing conditions to provide a treated protein; and
(b) conjugating said treated protein to a label moiety to provide said indirectly labelled protein conjugate, said label moiety comprising a carrier molecule coupled to one or more detectable labels.

2. The process according to claim 17 wherein said denaturing conditions comprise treatment of said protein with a protein denaturing agent.

3. The process according to claim 2, further comprising submitting the treated protein to a purification or dilution step to reduce the concentration of the protein denaturing agent prior to step (b).

4. The process according to claim 2, wherein said protein denaturing agent is urea.

5. The process according to claim 4, wherein said urea is at a concentration between about 5M and about 9M.

6. The process according to claim 1, further comprising submitting said treated protein to a reduction step prior to step (b).

7. The process according to claim 6, wherein said reduction step comprises treatment with dithiothreitol (DTT), dithioerythritol, mercaptoethanol, cysteine, or tris(2-carboxyethyl)phosphine (TCEP).

8. The process according to claim 1, wherein said carrier moiety comprises at least one detectable label.

9. The process according to claim 17 wherein said carrier molecule is a protein molecule.

10. The process according to claim 9, wherein said protein molecule is bovine serum albumin, thyroglobulin, haemocyanin, myosin, apoferritin, ovalbumin or α₂-macroglobulin.

11. The process according to claim 9, wherein said protein molecule is bovine serum albumin.

12. The process according to claim 1, wherein said protein comprises a viral antigen.

13. The process according to claim 1 wherein said protein comprises a hepatitis C viral antigen.

14. The process according to claim 13, wherein said hepatitis C viral antigen comprises a NS3 antigen.

15. The process according to claim 1, wherein said detectable label is a chemiluminescent label.

16. The process according to claim 15, wherein said chemiluminescent label is an acridinium ester.

17. A protein conjugate prepared by the process of claim 1.

18. The protein conjugate according to claim 17, wherein said carrier molecule is a protein molecule.

19. The protein conjugate according to claim 18, wherein said protein molecule is bovine serum albumin, thyroglobulin, haemocyanin, myosin, apoferritin, ovalbumin or α₂-macroglobulin.

20. The protein conjugate according to claim 18, wherein said protein molecule is bovine serum albumin.

21. The protein conjugate according to claim 17, wherein said protein comprises a viral antigen.

22. The protein conjugate according to claim 17, wherein said protein comprises a hepatitis C viral antigen.

23. The protein conjugate according to claim 22, wherein said hepatitis C viral antigen comprises a NS3 antigen.

24. The protein conjugate according to claim 17, wherein said detectable label is a chemiluminescent label.

25. The protein conjugate according to claim 24, wherein said chemiluminescent label is an acridinium ester.

26. A method of detecting a target analyte in a sample comprising utilizing the protein conjugate of claim 17, wherein said protein specifically binds to said target analyte.

27. The method according to claim 26, comprising the steps of: contacting said sample with the protein conjugate under conditions that allow the binding of the assay conjugate to the target analyte to form a complex, and detecting the complex as an indication of the presence of target analyte in the sample.

28. The method according to claim 27, comprising the steps of: contacting an immobilized capture agent which specifically binds to the target analyte with said sample under conditions that allow the binding of the target analyte to the capture agent to form a first complex; contacting said first complex with the assay conjugate under conditions that allow binding of the assay conjugate to the target analyte such that a second complex comprising the binding member, target analyte and assay conjugate is formed, and detecting the second complex as an indication of the presence of target analyte in the sample.

29. The method according to claim 26, wherein said protein comprises a viral antigen and said target analyte is a viral antibody.

30. The method according to claim 29, wherein said protein comprises a hepatitis C viral antigen and said target analyte is a hepatitis C virus antibody.

31. The method according to claim 30, wherein said hepatitis C viral antigen comprises a NS3 antigen and said hepatitis C virus antibody is an antibody against NS3.

32. A kit comprising the protein conjugate according to claim 17 and optionally instructions for use.

33. A protein conjugate comprising a hepatitis C virus NS3 antigen conjugated to a label moiety, said label moiety comprising a carrier moiety coupled to at least one detectable label.

34. The protein conjugate according to claim 33, wherein said protein conjugate is produced by the process according to claim 1.

35. The protein conjugate according to claim 33, wherein said label moiety is conjugated to said NS3 antigen via a linker.

36. The protein conjugate according to claim 33, wherein said carrier moiety is bovine serum albumin.

37. The protein conjugate according to claim 33, wherein said at least one detectable label is an acridinium ester.

38. The protein conjugate according to claim 33, wherein said NS3 antigen comprises SEQ ID NO:1.
